(19) United States
(12) Plant Patent Application Publication
(10) Pub. No.: US 2003/0135903 P1
(43) Pub. Date: Jul. 17, 2003

(54) Clementine tree named 'Clemenpons'

(76) Inventor: Juan Pons Bolta, Castellon (ES)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 10/047,911

(22) Filed: Jan. 15, 2002

(51) Int. Cl. A01H 5/00

(52) U.S. Cl. PLT/202

(57) ABSTRACT

A new and distinct Citrus reticulata tree with large, early ripening fruit.

### BACKGROUND OF THE INVENTION

The present invention comprises a new and distinct cultivar of clementine tree botanically known as Citrus reticulata, and herein referred to by its cultivar name ‘Clemenpons’.

The new cultivar was discovered in 1964 as a seedling in Pego, Aligante, Spain.

The new cultivar was first asexually reproduced in 1985 in Pego (Valencia), Spain by grafting budwood onto citrange rootstock. Subsequent grafting of budwood onto citrange rootsock in Moncado (Valencia), Spain has shown the features of the new cultivar to be stable and reproduce true to type in successive propagations.

The following traits are determined to be basic characteristics of the new cultivar which in combination distinguish this clementine tree as new and distinct.

1. Earlier fruit maturity then Clemenules.

2. Fruit characteristics similar to Clemenules; superior fruit quality to Arrufatina Clementine.

3. Tree characteristics similar to Clemenules; less vigorous growth than Fina (Commune) Clementine.

4. Fruit internal maturity and rind colors up to 3 weeks earlier than Clemenules.

5. Larger fruit than Fina (Commune) Clementine.

### DESCRIPTION OF THE DRAWINGS

The accompanying photographic drawings illustrate the new cultivar with the color being as true as possible with color illustrations of this type. The photographs were taken under natural light conditions.

FIG. 1 shows the tree;

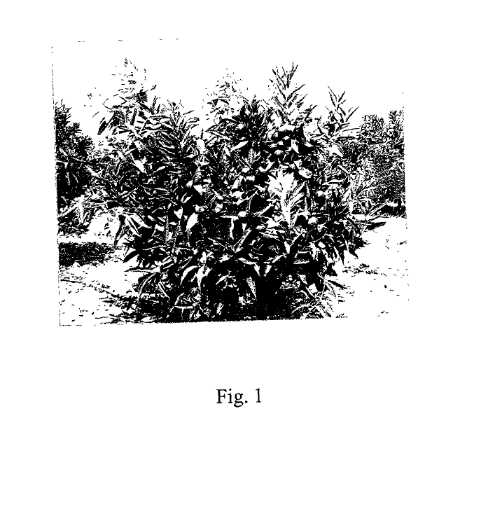

FIG. 2 shows fruit of the new variety; and

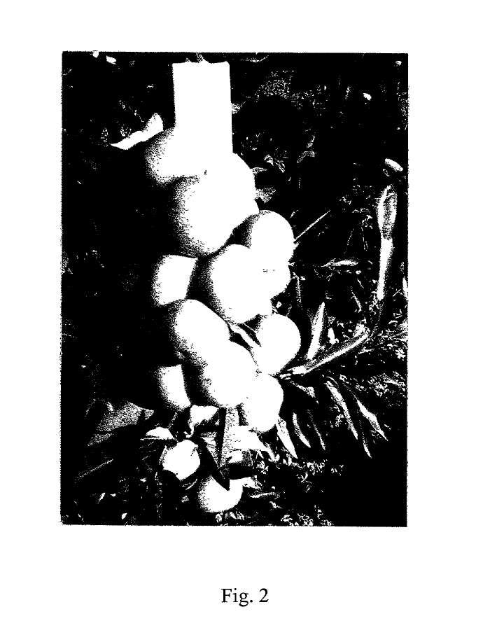

FIG. 3 shows color indices during opening of the fruit.

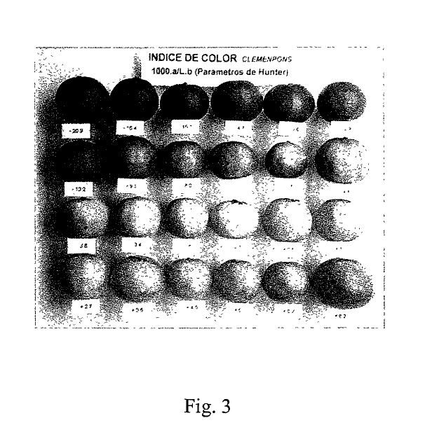

### DESCRIPTION OF THE PLANT

The following description sets forth the characteristics of the new cultivar. The data which defines these characteristics were collected from asexual reproductions by grafting of budwood onto rootstock. The plant history was taken on a 5 year old tree. In the following description, color references are made to the R.H.S. Colour Chart of The Royal Horticultural Society of London.

Classification:

Botanical.—Citrus reticulate.

Commercial.—Clementine tree.

Tree:

Growth rate.—Medium vigor; similar to Clemenules.

Overall shape.—Spreading.

Height.—Up to 12 feet, if allowed.

Spread.—Depending on spacing, up to 12 feet.

Preferred soil type.—Varies with rootstock type; loamy.

Foliage:

Shape.—Overall: Lanceolate. Base: Obtuse. Tip: Acute.

Size.—Length: 61 mm. Width: 20 mm.

Color.—Upper surface: Green Group 132A. Lower surface: Green Group 132B.

Marginal form.—Entire.

Petiole size.—Diameter: 1.2 mm. Length: 6 mm.

Scent.—Pleasant.

Surface texture.—Medium roughness.

Flowers:

Blooming period.—April in Moncada (Valencia), Spain.

Number of petals.—5.

Number of sepals.—5.

Petal size.—Length: 12 mm. Width: 5 mm.

Pedicel length.—4 mm.

Overall size.—Bud: Length: About 3 mm. Diameter: About 2.5 mm. Opened flower: Length: About 5 mm. Diameter: About 2.5 mm.

Petal color.—White Group 155B.

Fruit:

Maturity date.—Mid-October in Moncada (Valencia), Spain.

Weight.—70 to 120 g.

Shape.—Overall: Usually flat, depending on climate. Base: Slightly indented. Apex: Neck/nipple develops only in cold inland areas. Diameter: 55-65 mm. Height: 40-50 mm. Furrows: None.

Rind:

Surface texture.—Smooth; slightly pebbly.

Color.—Between Red-Orange Groups 28A and 28B.

Thickness.—2 mm.

Adherence to flesh.—Adheres tightly until mature.

Ease of peeling.—Very easy when mature.

Oil glands.—Even; not protruding.

Scent.—Mandarin-like.

Flesh:

Axis.—Straight.

Puffing.—Occurs when over mature.

Number of segments.—9-10.

Separability of segments.—Easily.

Pulp.—Color: Orange Group 28C. Texture: Tender.

Vesicles.—Shape: Tapered. Size: 2-4 mm.

Juice:

Relative amount in fruit.—46-50%.

Color.—Orange Group 28C.

Aroma.—Tangerine.

Flavor.—Tangerine-like.

Total soluble solids.—10-13° Brix.

Acid.—0.8-1.1%.

Seeds: Seedless under non-pollinating conditions.

Persistence of fruit on tree: Hangs well.

Use: Fresh eating.

Keeping quality: 40-60 days after picking at 18° C.

Shipping quality: Good, if adhere to protocols.

Persistence to diseases/pests: No unusual susceptibility to diseases or pests has been noted to date.

I claim:

1. A new and distinct variety of Citrus reticulata plant substantially as shown and described.
